(19) United States
(12) Patent Application Publication
(10) Pub. No.: US 2024/0139293 A1
(43) Pub. Date: May 2, 2024

(54) Compositions and Methods for Enhancing Anti-Viral Therapies

(71) Applicant: XORTX THERAPEUTICS, INC., Calgary (CA)

(72) Inventor: Allen DAVIDOFF, Calgary (CA)

(21) Appl. No.: 18/280,319

(22) PCT Filed: Mar. 7, 2022
(86) PCT No.: PCT/IB2022/000219

§ 371 Date: Sep. 5, 2023

Related U.S. Application Data
(60) Provisional application No. 63/157,032, filed on Mar. 5, 2021.

(51) Int. Cl. A61K 38/44; A61K 31/198; A61K 31/375; A61K 38/21; A61K 45/06

(52) U.S. Cl. CPC A61K 38/44; A61K 31/198; A61K 31/375; A61K 38/21; A61K 45/06; C12Y 107/03003

(57) ABSTRACT

The present disclosure is directed to the use of a uric acid lowering agent (UALA) to increase the effectiveness of interferon in a subject with a viral infection. The UALA may be administered in combination with interferon in a subject undergoing interferon therapy.

### FIELD

The invention relates to methods, compositions, and uses, of uric acid lowering agents (UALA) for decreasing serum or tissue uric acid concentration and/or inhibiting uric acid production by xanthine oxidase activity or uric acid reuptake by the kidneys in the setting of viral infection as a means of decreasing negative inhibition of Interferon therapy in animals and humans.

### BRIEF DESCRIPTION OF THE DRAWINGS

FIG. 1: Hyperuricemia is increased early in hospitalized patients infected with COVID-19.

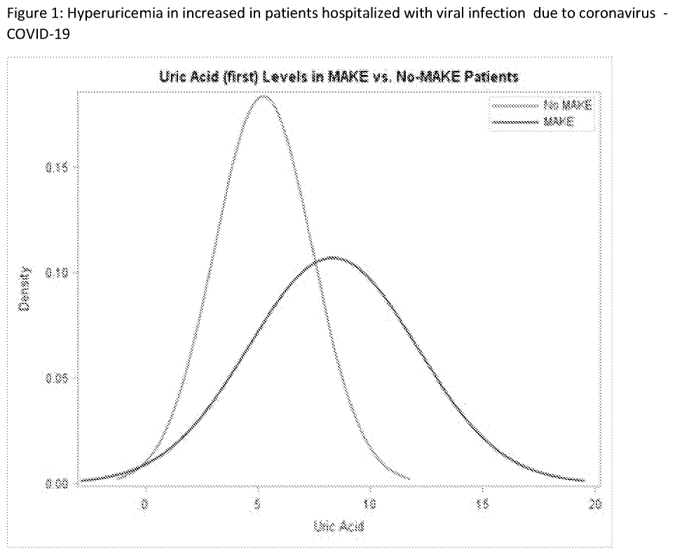

FIG. 2: Severity of acute kidney injury is associated dose dependently with serum uric acid concentration.

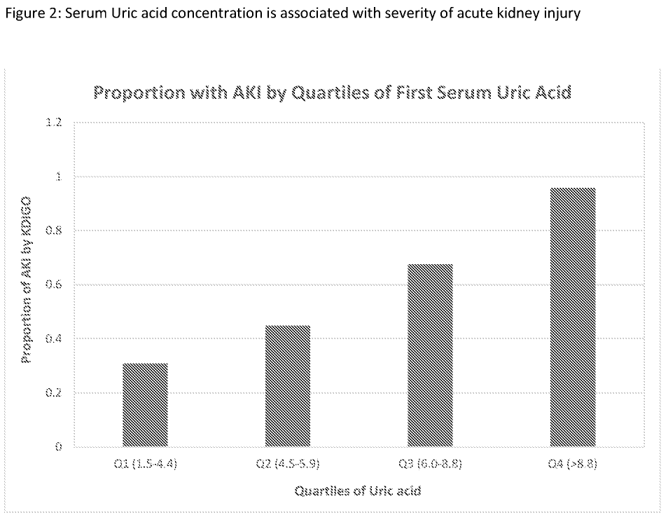

FIG. 3: Uric acid is dose dependently associated with increase procalcitonin concentration—a marker of inflammatory state and bacterial infection/sepsis.

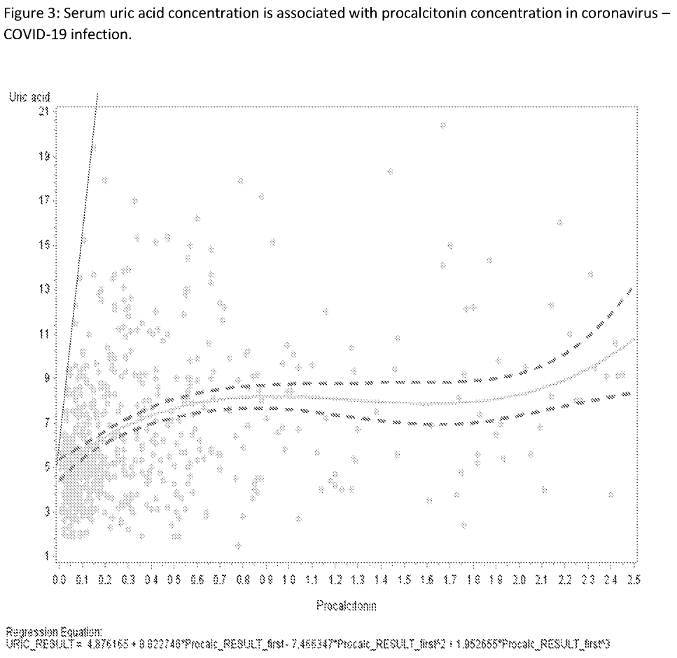

### DETAILED DESCRIPTION

### Overview

Respiratory Viral infections are primarily initialized as an infection of the respiratory tract. Examples of viruses that infect the respiratory tract are rhinoviruses, influenza viruses (during annual winter epidemics), parainfluenza viruses, respiratory syncytial virus (RSV), enteroviruses, coronaviruses, and certain strains of adenovirus are the main causes of viral respiratory infections.

Coronaviruses and specifically COVID-19 represent new emerging viruses affecting humans and is one type of virus amongst a family of viruses that effect a variety of species. Coronavirus infection in humans is characterized by a broad array of physiologic and anatomical abnormalities that can result in an acute or chronic condition, for example, including altered glucose disposition, hypertension, retinopathy, abnormal kidney function, abnormal central nervous system function, abnormal cardiac function, abnormal liver function, abnormal platelet activity, abnormal pancreatic function aberrations involving large, medium and small sized vessels, chronic fatigue, rhabdomyolysis, and other co-morbidities and death.

Coronavirus infection and specifically COVID-19 infection has been described initiating in the respiratory tract and involving, sinus, trachea, bronchi and lung function leading to lung injury, hypoxia, shortness of breath, pulmonary embolism. Whether serially or in parallel, blood vessel function, endothelial cell infection, kidney, gastrointestinal, neurological, cardiovascular, pancreatic, injury, skeletal muscle injury and susceptibility to bacterial infection have been described. In addition, associated with coronavirus infection and specifically COVID-19 rhabdomyolysis, and/or hyperactive catabolic syndrome and or acute respiratory distress syndrome and aberrant cytokine expression has been described. Increased cytokine expression, inflammatory status, oxidative stress and a pro-coagulative state have been described.

A class of signaling proteins—interferons—are employed as anti-viral defensive to limit viral infection spread, severity and alert cells of neighboring tissues to prepare to defend against potential viral infection. It has been reported that hyperuricemia is a factor that can suppress interferon potency. Individuals with obesity, hypertension, metabolic syndrome, insulin resistance, pre-diabetes, diabetes and with chronic kidney disease have a higher incidence of hyperuricemia and endothelial dysfunction.

The achievement of early virologic response (EVR) implies a high possibility of sustained viral response (SVR) [10, 12, 13]. In addition, diverse viral or host factors have been reported to affect the response to the conventional treatment [10, 14-16]. A number of studies have been performed to find new predictors for treatment response. Insulin resistance, the main pathophysiology for metabolic syndrome, was reported to reduce the achievement of SVR in patients with chronic HCV [3, 17, 18]. Uric acid, which is synthesized at the last step of purine decomposition, is metabolized in the liver, and serum level tends to be elevated in patients with obesity, metabolic syndrome, diabetes and chronic kidney disease [19]. Indeed, uric acid concentration has been identified as a predictor of interferon therapy effectiveness, using a measure of sustained virological response (SVR). Soo K_et al_Clinical implication of serum uric acid in pegylated interferon and ribavirin combination therapy for chronic hepatitis C infection_Korean J Intern Med_32_1010_2017_kjim-2016-405

Viral Infection—Viral infection can induce two types of immune response—innate immune response against a virus involving the synthesis of protein hormones—cytokines—and the stimulation of “natural killer” lymphocytes. If innate response is insufficient to suppress or prevent an infection and the infection proceeds more than the first few rounds of infection, the “adaptive immune response”, initiates. The adaptive response has two fundamental components, 1/Humoral response involving the synthesis of virus-specific antibodies by B lymphocytes and 2/the cell-mediated response involving the genesis of specific cytotoxic T lymphocytes that kill infected cells. These components of the adaptive immune response result in the production of long-lived “memory cells” that permit a more rapid response (immunity) to subsequent infections by the same or related viruses.

The lytic cycle is one of the two cycles of viral reproduction, the other being the lysogenic cycle. The lytic cycle results in the destruction of the infected cell and its membrane.

The lytic cycle: The normal process of viral reproduction involving penetration of the cell membrane, nucleic acid synthesis, and lysis of the host cell.

The lysogenic cycle: A form of viral reproduction involving the fusion of the nucleic acid of a bacteriophage with that of a host, followed by proliferation of the resulting prophage.

### Lytic Viruses∴cfDNA

Lytic viruses release viral particles and cellular contents into the circulation, including cell free DNA (cfDNA). When present cfDNA is rapidly degraded to uric acid for secretion and when sufficiently concentrated can saturate serum and promote monosodium urate crystal formation.

Studies of the relationship between viral, fungal and bacterial infection show that serum DNA (DNAemia) as a result of viral infection predicts increased fungal and bacterial infection. {Walton A H_Reactivation of Multiple Viruses in Patients with Sepsis_PlosOne_Vol9_Issue6_98819_2014}. This finding suggest that lytic virus and viral load during and infection, may contribute to increase DNAemia, subsequently increase serum uric acid and an immunosuppressive state. A recent study by Murray et al, 2021, shows that uric acid lowering agents such as probenecid can decrease viral load due to Coronavirus. {Murray J_et al_Probenecid Inhibits SARS-CoV-2 replication in vivo and invitro_Nature_September 2021_541598-021-97658-w} Decreased viral load would tend to decrease DNAemia and so, lower serum uric acid concentrations would be expected. Levels. Results of this study suggest that both decreasing uric acid uptake with a uricosuric drug such as probenecid may decrease an immunosuppressive state that results from high serum uric acid levels. Indeed, uricase digestion of uric acid into allantoin or inhibition of xanthine oxidase production of uric acid or its drugs such as sGPLT2 inhibitors that suppress xanthine oxidase expression could conceptually attenuate immunosuppression due to hyperuricemia.

Sepsis is a systemic inflammatory syndrome caused by massive microbial infections and is a major cause of death worldwide. Sepsis is defined as an “organ dysfunction caused by a dysregulated host response to infection,” while septic shock is associated to a greater mortality risk, caused by “underlying circulatory, cellular and metabolic abnormalities” (1, 2). Although survival of sepsis patients with overwhelming pro-inflammatory responses is greatly improved in intensive care units (ICU), most patients develop delayed sepsis with severely suppressed immune responses and succumb to secondary infections (3, 4).

Serum Uric Acid (SUA) is associated with CRP, IL-1, IL-6, TNF-a and procalcitonin in COVID-19 infections. Increasing procalcitonin is associated with increase SUA suggesting that uric acid may mediate increased susceptibility to secondary bacterial infection and subsequently sepsis.

Viruses such as Coronaviruses and specifically COVID-19 infect individuals by attaching to the host cell using the host cell receptor. In the case of COVID-19, tissues expressing ACE2 are most susceptible to infection as this respiratory virus infects primary tissues, then secondary tissues. For example, pulmonary tissue infection leading to secondary infection of vascular, kidney, heart, brain, pancreas and other tissues.

Individuals with pre-existing conditions such as obesity, hypertension, metabolic syndrome, insulin resistance, diabetes, and kidney disease are reported to have increased risk of more severe viral infections, health consequences of COVID-19. Male gender has also been reported to be a risk factor for more serious viral infection. Individuals with obesity, hypertension, metabolic syndrome, insulin resistance, diabetes, and kidney disease often have coincident hyperuricemia and endothelial dysfunction.

As part of the life cycle of a virus, production of new viral particles within and lysis of host cell results in release of viral particles and release of unused cellular components into the interstitial space and/or circulation dependent upon the location into the infected host cell. Cellular contents released by viruses can activate and enhance inflammatory responses and specifically cell free DNA (cf DNA). Uric acid is synthesized mainly in the liver, intestines and other tissues such as muscles, kidneys and the vascular endothelium as the end product of an exogenous pool of purines, derived largely from animal proteins. In addition, live and dying cells degrade their nucleic acids, adenine and guanine into uric acid.

Hyperuricemia has been reported to increase and modulate worse endothelial dysfunction. Uric acid lowering agents have been reported to decrease or reverse endothelial dysfunction. Frequently, hyperuricemia, endothelial dysfunction, obesity, hypertension, metabolic syndrome, insulin resistance, diabetes, and kidney disease have been associated with increased expression of ACE2 receptors within affected tissues. Indeed hyperuricemia, has been implicated as augmenting systemic and tissue specific inflammation, vascular injury, and kidney injury.

Recent publications have suggested that the interleukin (IL) response is increased by IL-1B expression and counterbalanced and suppressed by IL-1RA. Hyperuricemia has been shown to decrease the suppressive effect of IL-1RA inhibition, thereby permitting excessive IL-1a and IL-1b signaling to escalate in the absence of this inhibition and excessive cytokine meditated inflammation.

During viral infections such as those attributable to lytic virus or coronavirus or COVID-19 infection, and therapeutically decreasing and suppressing uric acid levels would potentially restore control and suppression of runaway interleukin signaling and inflammation. This invention describes compositions and methods of uric acid lowering (UALA) as a means of suppressing uncontrolled cytokine signaling and resulting inflammation, hypercatabolic state, and hyper coagulative state and attenuating the health consequences of viral infections in subjects during viral, coronavirus or COVID-19 infection.

In another embodiment of this invention, the hyperuricemic state—serum uric acid greater than 5.5 mg/dL—represents an environment that potentially permits excess inflammatory response by inhibiting IL-1RA counter-inhibition of IL signaling. The invention in this scenario has implications for viral and bacterial sepsis and susceptibility to bacterial sepsis secondary to viral infection. The utility of uric acid lowering as a therapy to restore IL-1Ra inhibition of IL signaling and therapeutic effectiveness of interferon.

Hyperuricemia, has been associated with increased expression of cytokines such as IL-1, IL-6, PGE2, TNF-a, c-Reactive protein (CRP). These results suggest that uric acid contributes to systemic inflammation in humans and are in agreement with experimental data showing that uric acid triggers sterile inflammation. Lyngdoh T, et al, Elevated Serum Uric Acid is Associated with High Circulating Inflammatory Cytokines in the Population-Based Colaus Study, PLoS Ine 6(5)e19901, 2011 Sterile-inflammation is a form of pathogen-free inflammation caused by mechanical trauma, ischemia, stress or environmental conditions such as ultra-violet radiation. These damage-related stimuli induce the secretion of molecular agents collectively termed danger-associated molecular patterns (DAMPs).

In response to viral infection, mammalian cells respond with increased interferon expression. In health interferons are a class of signaling proteins synthesized and released by host cells in response to the presence of a variety of viruses. A host cell infected by a virus, can use this release of signaling interferon molecules to increase anti-viral defenses of other cells. Interferons (IFNs) are a class of proteins known as cytokines, molecules used for communication between cells to trigger protective defenses of the immune system that help eradicate pathogens. Parkin J, Cohen B (June 2001). “An overview of the immune system”. Lancet. 357 (9270): 1777-89.

Inflammatory responses are critical because they not only alert cells to initiate an effective immune response during infections but also initiate wound repair and healing programs. In contrast, excessive unresolved inflammation can lead to tissue damage and disease. Each type of inflammatory response is comprised of unique sets of molecular events, lipid mediators, cytokines and specialized cell types that initialize inflammation, followed by an equally tuned set of steps to ensure resolution of inflammation. Innate cytokines Interluekin-1 (IL-1) and type 1 interferons (IFN-1) are the foundation of two major types of inflammatory responses that can antagonize each other and in the setting of hyperuricemia may permit enhanced acute respiratory distress syndrome (ARDS), uncontrolled cytokine expression—“cytokine storm” and provide the establishment and promote sepsis.

IL-1 is a pro-inflammatory cytokine and a pyrogen, and mediates highly inflammatory responses via two cytokine species IL-1a and IL-1B. Both species can be expressed separately by most cell types and generate biological responses in many body systems. Excessive IL-1 overproduction is highly detrimental and contributes to a number of diseases. IL-1 therefore is extensively regulated and the margin for clinical benefit and undesirable pathogenic effect for IL-1 has been described as exceedingly narrow. Mayer-Barber et al, Interleukin-1 and type 1 IFN, Cellular and Molecular Biology, (14) 22, 2107

IL-1a and IL-1B signal through an IL-1 receptor (IL-1R1), and a third ligand for the receptor IL-1Ra, is a naturally occurring specific IL-1R1 receptor antagonist and prevents IL-1a and IL-1B mediated signaling. A second IL-1R chain, the IL-1R11, represents a decoy receptor that also acts to limit IL-1 driven responses.

Several other studies describe a downregulation of IL-1Ra in context of gout. GM-CSF treated neutrophils produce IL-1a, IL-113 and IL-1Ra. MSU crystals cause a drastic reduction in the IL-1Ra to total IL-1 ratio resulting in a pro-inflammatory imbalance Roberge C J et al, 1994. Soluble urate also leads to a decrease in IL-1Ra in human monocytes upon stimulation [16]. These studies suggest IL-1Ra insufficiently compensates for pro-inflammatory cytokines induced in hyperuricemia. When interpreted together these studies suggest that hyperuricemia may permit uncontrolled hyperinflammatory response to viral and bacterial infections such as observed in ARDS, hypercatabolic state, cytokine storm, or sepsis. C. J. Roberge, R. de Medicis, J. M. Dayer, et al. Crystal-induced neutrophil activation. V. Differential production of biologically active IL-1 and IL-1 receptor antagonist J Immunol, 152 (1994), pp. 5485-5494; T. O. Crisan, M. C. P. Cleophas, B. Novakovic, et al. Uric acid priming in human monocytes is driven by the AKT-PRAS40 autophagy pathway Proc Natl Acad Sci USA, 114 (2017), pp. 5485-5490

It follows that treating hyperuricemia with a uric acid lowering agent or agents may restore IL-1Ra antagonism of inflammation.

IL-1 is most widely studied and implicated in host resistance to acute bacterial infections, such as Staphylococcus aureus, where rapid inflammatory responses and IL-1 induced chemokines are required for optimal neutrophil-dependent control. Although IL-1 mediated host resistance most commonly in bacterial infections, IL-1 signaling can also protect against viral infections including influenza. Recent studies using human pulmonary microvascular endothelial cells showed that IL-1B secreted by the endothelial cells contributes to influenza-induced inflammation. In hyperuricemia, where IL-1Ra responsiveness is attenuated, excessive inflammatory response to viral/coronavirus/COVID-19 may make some individuals more susceptible to worse infection and health consequences of that infection.

Although, IL-1 protects most commonly against bacterial infections, type 1 Interferons (type 1 IFN) belong to a family of cytokines that are specialized to be highly protective against viral infections. Most cell types express type 1 IFN's through cytosolic receptors upon recognition of viral RNA or DNA.

Type I IFNs are the prototypical cytokines associated with control of viral infections as they successfully restrict viral replication by an acute induction of specific sets of several hundreds of ISGs inside infected cells that can directly interrupt viral gene transcription and translation. These genes are induced by type I IFNs in response to innate viral recognition Interleukin-1 and type I IFN and also promote an antiviral state in bystander cells that limits viral spread.

High serum uric acid concentrations have been associated with pro-inflammatory signals increased prostaglandin E2 (PGE2) and IL-1 as well decreased anti-inflammatory IL-Ra expression. IL-1 has been reported to inhibit IFN-B production, thereby acting to suppress the anti-inflammatory effects of type I Interferons—a potential “interferon resistant” state. PGE2 has been shown to suppress type 1 IFN production in mice and during influenza infection. Thus, studies in animals revealed that IL-1 can potentially antagonize type 1 IFN responses by directly regulating both transcription and translation of IFN-β via induction of PGE2.

Type I IFN's can also inhibit IL-1 levels and increase IL-1Ra levels—an anti-inflammatory action. Importantly, both IFNa—alpha and IFN-B—beta can suppress II-1a and IL-1B transcription and translation in various cell types. However, this anti-inflammatory action of IFN's has a cost as it has been reported that susceptibility to bacterial infections can be increased. IFN action is thought to potentially induce anti-inflammatory action by increasing IL-1Ra ad IL-10 which in turn can inhibition IL-1 signaling effects. This antagonism of IL-1 does not seem to be limited to type I IFN's but has been reported for type II IFN IFN-gamma and recently type III IFN's as well.

The embodiments provided in this disclosure are based on the discovery that animals including humans in a state of hyperuricemia (serum uric acid >5.5 mg/dL), have increased IL-1 activity, increased inflammatory state and “interferon resistant” state. This Interferon resistant state can suppress the IFN mediated antiviral immune response and increase susceptibility to more severe viral infection. Accordingly, certain embodiments relates to use of uric acid lowering agents to reverse endothelial dysfunction, reverse the “interferon resistant” state and permit increased antiviral responsiveness in and animal or with administration of Interferon of type I, II, or III which viaUALAs known action of decreasing circulating uric acid.

In another embodiment, decreasing serum or tissue uric acid levels would decrease expression of IL-1 and PGE2, and increase expression of IL-1Ra expression thereby permitting the host to mount an anti-bacterial response. Primary viral sepsis and secondary bacterial sepsis would be more likely when hyperuricemia is suppressing the anti-bacterial IL-1 signaling cytokines which in turn suppress the IFN anti-viral response. Lowering uric acid concentration and/or preventing production of uric acid in a subject with a viral infection will reverse this negative feedback on both systems and therapeutically treat and prevent viral and bacterial sepsis and the health consequences of viral infection.

### Definitions

“Reduction” of a symptom(s) means a decreasing of the severity or frequency of the symptom(s), or elimination of the symptom(s).

“Administering” or “administration of” a drug or therapeutic pharmaceutical composition to a subject by any method known in the art includes both direct administration, including self-administration (including oral administration or intravenous, subcutaneous, intramuscular or intraperitoneal injections, rectal administration by way of suppositories) or administration by any route or method that delivers a therapeutically effective amount of the drug or composition to the cells or tissue to which it is targeted (e.g. pulmonary administration).

As used herein, the terms “co-administered, “co-administering,” or “concurrent administration”, when used, for example with respect to administration of an active agent with another active agent, or a conjunctive agent along with administration of an active agent refers to administration of the active agent and the other active agent and/or conjunctive agent such that both can simultaneously achieve a physiological effect. The two agents, however, need not be administered together. In certain embodiments, administration of one agent can precede administration of the other, however, such co-administering typically results in both agents being simultaneously present in the body (e.g. in the plasma) of the subject. Co-administering includes providing a co-formulation (in which the agents are combined or compounded into a single dosage form suitable for oral, subcutaneous or parenteral administration).

“Preventing a disease” includes, but is not limited to, preventing the disease from occurring in a subject that may be predisposed to the disease (or disorder), but has not yet been diagnosed as having the disease; inhibiting the disease, for example, arresting the development of the disease; and/or delaying disease onset. An example is increasing interferon effectiveness in a subject at risk of getting Covid and who has an elevated uric level of 5.5 mg/dl.

A “therapeutically effective amount” of a uric acid lowering agent (UALA), interferon, or other agent or pharmaceutical composition comprising such agents is an amount that achieves the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

A “prophylactically effective amount” of a drug is an amount of a drug that, when administered to a subject, will have the intended prophylactic effect, e.g., preventing or delaying the onset (or reoccurrence) of the disease or symptoms, or reducing the likelihood of the onset (or reoccurrence) of the disease or symptoms. The full prophylactic effect does not necessarily occur by administration of one dose and may occur only after administration of a series of doses. Thus, a prophylactically effective amount may be administered in one or more administrations. For diabetes, a therapeutically effective amount can also be an amount that increases insulin secretion, increases insulin sensitivity, increases glucose tolerance, or decreases weight gain, weight loss, or fat mass.

An “effective amount” of an agent is an amount that produces the desired effect.

By “pharmaceutically acceptable” is meant that the carrier, diluent or excipient must be compatible with the other ingredients of the formulation and not deleterious to the recipient thereof.

“Treating” a disease, disorder or condition in a patient refers to taking steps to obtain beneficial or desired results, including clinical results. For purposes of this invention, beneficial or desired clinical results include, but are not limited to reduction, alleviation or amelioration of one or more symptoms of the disease; diminishing the extent of disease; delaying or slowing disease progression; amelioration and palliation or stabilization of the disease state or symptoms and its complications.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

Anti-inflammatory agents include but are not limited to steroids, e.g., budesonide, nonsteroidal anti-inflammatory agents, e.g., aminosalicylates (e.g., sulfasalazine, mesalamine, olsalazine, and balsalazide), cyclooxygenase inhibitors (COX-2 inhibitors, such as rofecoxib, celecoxib), baricitinib, diclofenac, etodolac, famotidine, fenoprofen, flurbiprofen, ketoprofen, ketorolac, ibuprofen, indomethacin, meclofenamate, mefenamic acid, meloxicam, nambumetone, naproxen, oxaprozin, piroxicam, salsalate, sulindac, tolmetin.

The term “interferon” has its commonly understood meaning pertaining to an antiviral compound. In health interferons are a class of signaling proteins synthesized and released by host cells in response to the presence of a variety of viruses. A host cell infected by a virus, can use this release of signaling interferon molecules to increase anti-viral defenses of other cells. Interferons (IFNs) are a class of proteins known as cytokines, molecules used for communication between cells to trigger protective defenses of the immune system that help eradicate pathogens. Parkin J, Cohen B (June 2001). “An overview of the immune system”. Lancet. 357 (9270): 1777-89. Interferons can also activate immune cells, such as natural killer cells (NK), and macrophages (MO), by increasing antigen presentation and expression of major histocompatibility complex MHC) antigens.

More than twenty distinct IFN genes and proteins have been identified in animals, including humans. They are typically divided among three classes: Type I IFN, Type II IFN, and Type III IFN. IFNs belonging to all three classes are important for fighting viral infections and for the regulation of the immune system.

Based on the type of receptor through which they signal, human interferons have been classified into three major types.
Interferon type I: All type I IFNs bind to a specific cell surface receptor complex known as the IFN-α/β receptor (IFNAR) that consists of IFNAR1 and IFNAR2 chains. de Weerd N A, Samarajiwa S A, Hertzog P J (July 2007). “Type I interferon receptors: biochemistry and biological functions”. The Journal of Biological Chemistry. 282 (28): 20053-7 The type I interferons present in humans are IFN-α, IFN-β, IFN-ε, IFN-κ and IFN-ω. Liu Y J (2005). “IPC: professional type 1 interferon-producing cells and plasmacytoid dendritic cell precursors”. Annual Review of Immunology. 23: 275-306] In general, type I interferons are produced when the body recognizes a virus that has invaded it. They are produced by fibroblasts and monocytes. However, the production of type I IFN-α is inhibited by another cytokine known as Interleukin-10. Once released, type I interferons bind to specific receptors on target cells, which leads to expression of proteins that will prevent the virus from producing and replicating its RNA and DNA. Levy D E, Marié I J, Durbin J E (December 2011). “Induction and function of type I and III interferon in response to viral infection”. Current Opinion in Virology. 1 (6): 476-86. Overall, IFN-α can be used to treat hepatitis B and C infections, while IFN-β can be used to treat multiple sclerosis. Parkin J, Cohen B (June 2001). “An overview of the immune system”. Lancet. 357 (9270): 1777-89.
Interferon type II (IFN-γ in humans): This is also known as immune interferon and is activated by Interleukin-12. Parkin J, Cohen B (June 2001). “An overview of the immune system”. Lancet. 357 (9270): 1777-89. Type II interferons are also released by cytotoxic T cells and type-1 T helper cells. However, they block the proliferation of type-2 T helper cells. The previous results in an inhibition of Tₕ2 immune response and a further induction of Tₕ1 immune response. Kidd, P (2003). “Th1/Th2 Balance: the hypothesis, its limitations, and implications for health and disease”. Alternative Medicine Review. 8 (3): 223-46. IFN type II binds to IFNGR, which consists of IFNGR1 and IFNGR2 chains. Parkin J, Cohen B (June 2001). “An overview of the immune system”. Lancet. 357 (9270): 1777-89.
Interferon type III: Signal through a receptor complex consisting of IL10R2 (also called CRF2-4) and IFNLR1 (also called CRF2-12). Although discovered more recently than type I and type II IFNs, Kalliolias G D, Ivashkiv L B (2010). “Overview of the biology of type I interferons”. Arthritis Research & Therapy. 12 Suppl 1 (Suppl 1): S1 recent information demonstrates the importance of Type III IFNs in some types of virus or fungal infections. Vilcek, Novel interferons, Nature Immunol. 4, 8-9. 2003 Hermant P, Michiels T (2014). “Interferon-λ in the context of viral infections: production, response and therapeutic implications”. Journal of Innate Immunity. 6 (5): 563-74; Espinosa V, Dutta O, McElrath C, Du P, Chang Y J, Cicciarelli B, Pitler A, Whitehead I, Obar J J, Durbin J E, Kotenko S V, Rivera A (October 2017). “Type III interferon is a critical regulator of innate antifungal immunity”. Science Immunology. 2 (16) In general, type I and II interferons are responsible for regulating and activating the immune response. Parkin J, Cohen B (June 2001). “An overview of the immune system”. Lancet. 357 (9270): 1777-89

Expression of type I and III IFNs can be induced in virtually all cell types upon recognition of viral components, especially nucleic acids, by cytoplasmic and endosomal receptors, whereas type II interferon is induced by cytokines such as IL-12, and its expression is restricted to immune cells such as T cells and NK cells.

Examples of interferon agents suitable for administration include, but are not limited to, What interferons are approved for marketing: interferon alfa-2a (Roferon-A)
interferon alfa-2b (Intron-A)
interferon alfa-n3 (Alferon-N)
peginterferon alfa-2b (PegIntron, Sylatron)
interferon beta-1a (Avonex)
interferon beta-1a (Rebif)
interferon beta-1b (Betaseron)
interferon beta-1b (Extavia)
interferon gamma-1b (Actimmune)
peginterferon alfa-2a (Pegasys ProClick)
peginterferon alfa-2a and ribavirin (Peginterferon)
peginterferon alfa-2b and ribavirin (PegIntron/Rebetol Combo Pack)
peginterferon beta-1a (Plegridy)
interferon alfacon-1 (Infergen has been discontinued in the US)

As used herein, the terms “comprises,” “comprising,” “containing,” “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “uric acid lowering agent” or “UALA” refers to a compound that reduces uric acid in a subject upon administration of the compound. Examples of UALAs include but are not limited a uricase (for example: Pegloticase or Rasburicase), uricosuric agents (for example, losartan, probenecid, benzbromarone, atorvastatin, fenfibrate, lesinurad, venurinad, sulfinpyrazone, or pyrazinamide), xanthine Oxido-Reductase inhibitors (allopurinol, febuxostat, TMX-049, oxypurinol, NC-2500, NC-2700, NMDA, etc), or agents that inhibit expression of xanthine oxidase such as an SGLT2 inhibitor (canagliflozin, dapagliflozin or empagliflozin), or agents that modify the ratio of XO to XDH. Indeed, because uric acid and oxypurinol are potentially building blocks for nucleic acids, the antiviral effect of oxypurinol may prove beneficial in coronavirus infections (Perez-Mazliah D et al, Allupurinol reduced antigen-specific and polyclonal activation of human T-cells, Frontiers in Immunology, September 2012).

Interleukin (IL), any of a group of naturally occurring proteins that mediate communication between cells. Interleukins regulate cell growth, differentiation, and motility. They are particularly important in stimulating immune responses, such as inflammation.

The human genome encodes more than 50 interleukins and related proteins. Brocker C, Thompson D, Matsumoto A, Nebert D W, Vasiliou V (October 2010). “Evolutionary divergence and functions of the human interleukin (IL) gene family”. Human Genomics. 5 (1): 30-55.

The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Pharmaceutical Formulations and Administration

The present disclosure also includes pharmaceutical compositions and formulations of UALA agents for use in conjunction with treating viral or bacterial infections and/or potentiating anti-viral activity of interferon. In typical embodiments, pharmaceutical compositions comprise therapeutically effective amounts of a UALA for lowering uric acid in a subject. Pharmaceutical compositions for use in the present methods include therapeutically effective amounts of one or more UALAs, in an amount sufficient to prevent or treat the diseases described herein in a subject, formulated for systemic administration. In an optional embodiment, UALA may be co-administered with at least one other active agent. The subject is preferably a human but can be non-human as well. A suitable subject can be an individual who is suspected of having, has been diagnosed as having, or is at risk of developing a viral and/or bacterial infection.

A composition comprising UALA can also include a pharmaceutically acceptable carrier. Such compositions may contain, for example, such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70% active ingredient.

The UALAs can also be mixed with diluents or excipients which are compatible and physiologically tolerable as selected in accordance with the route of administration and standard pharmaceutical practice. Suitable diluents and excipients are, for example, water, saline, dextrose, glycerol, or the like, and combinations thereof. In addition, if desired, the compositions may contain minor amounts of auxiliary substances such as wetting or emulsifying agents, stabilizing or pH buffering agents.

The formulations may also contain more than one active compound as necessary for the particular indication being treated, preferably those with complementary activities that do not adversely affect each other. Such molecules are suitably present in combination in amounts that are effective for the purpose intended.

The UALAs may be formulated for administration by any suitable means. For in vivo administration, the pharmaceutical compositions are preferably administered orally or parenterally, i.e., intraarticularly, intravenously, intraperitoneally, subcutaneously, or intramuscularly. In particular embodiments, the pharmaceutical compositions are administered intravenously or intraperitoneally by a bolus injection. Stadler, et al., U.S. Pat. No. 5,286,634. For the prevention or treatment of disease, the appropriate dosage will depend on the severity of the disease, whether the drug is administered for protective or therapeutic purposes, previous therapy, the patient's clinical history and response to the drugs and the discretion of the attending physician.

The resulting pharmaceutical preparations may be sterilized by conventional, well known sterilization techniques. The aqueous solutions can then be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the lipidic suspension may include lipid-protective agents which protect lipids against free-radical and lipid-peroxidative damages on storage. Lipophilic free-radical quenchers, such as α-tocopherol and water-soluble iron-specific chelators, such as ferrioxamine, are suitable.

The pharmaceutical compositions of this disclosure may be in a variety of forms, which may be selected according to the preferred modes of administration. These include, for example, solid, semi-solid and liquid dosage forms such as tablets, lozenge, pills, powders, liquid solutions or suspensions, suppositories, and injectable and infusible solutions. The preferred form depends on the intended mode of administration and therapeutic application. For oral administration, the UALA may be formulated as dispersible tablet, orally disintegrating tablet, effervescent tablet, chewable tablet, sprinkle granules, dry suspension or dry syrup for reconstitution, quick melt wafers, lozenge, or chewing gum.

The pharmaceutical compositions disclosed herein may, for example, be placed into sterile, isotonic formulations with or without cofactors which stimulate uptake or stability. The formulation is preferably liquid or may be lyophilized powder. This solution can be lyophilized, stored under refrigeration and reconstituted prior to administration with sterile Water-For-Injection (USP).

Formulations for parenteral administration include aqueous and non-aqueous sterile injection solutions, which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described.

It will be understood that the methods and uses of the disclosure may be employed prophylaxis as well as (more suitably) in the treatment of subjects suffering from a viral and/or bacterial infection before uric acid levels are elevated.

The disclosure has references to specific embodiments. It will, however, be evident that various modifications and changes may be made thereto without departing from the broader spirit and scope of the disclosure. The specification and drawings are, accordingly, to be regarded in an illustrative rather than a restrictive sense. The disclosure is illustrated herein by the experiments described above and by the following examples, which should not be construed as limiting. The contents of all references, pending patent applications and published patents, cited throughout this application are hereby expressly incorporated by reference. Although specific terms are employed, they are used as in the art unless otherwise indicated. Other information concerning formulations and dosing of UALAs is provided in US Publication No US20160038595 and WO/2007/018687 whose teachings are incorporated herein.

What is claimed is:

1. A composition comprising a uric acid lowering agent (UALA) or UALAs for use in increasing interferon effectiveness in a subject with a viral infection.

2. A composition of (1) whereby the composition is formulated for intravenous delivery.

3. A composition of (1) whereby the composition is formulated for oral, intravenous and/or intramuscular delivery, or a combination of routes.

4. A composition of 1, 2, or 3 that includes an oxygen radical scavenging agent such as vitamin C, N-acetyl cysteine.

5. A composition of 1, 2, 3 or 4 that include and organic base, and/or organic amino acid, such as arginine or lysine.

6. The composition of any of claims 1-5, wherein the viral infection is a respiratory viral infection.

7. The composition of claim 6, wherein the respiratory viral infection is by a coronavirus.

8. The composition of claim 7, wherein the coronavirus is Sars-Cov-2.

9. The composition of any of claims 1-8, wherein the subject is undergoing interferon administration.

10. The composition of any of claims 1-8, wherein the composition further comprises interferon.

11. A composition of uric acid lowering agent or agents or interferon or anti-inflammatory agent for use in decreasing systemic inflammation associated patients with coronavirus/COVID-19 infection.

12. A composition of uric acid lowering agent, and anti-inflammatory for use in treating or preventing viral or bacterial sepsis.

13. The composition of claim 12, wherein the bacterial sepsis is a secondary infection following a viral infection.

14. A composition of any of claims 1-8 for use in treating health consequences of hyperuricemia increasing severity of COVID-19 infection.

15. A method of enhancing the anti-viral effect of Interferon therapy during a viral infection comprising administering a therapeutically effective amount of a UALA.

16. The method of claim 15, further comprising co-administering a therapeutically effective amount of interferon.

17. The method of claims 15 and 16, wherein the UALA and interferon are co-administered in the same composition.

18. The method of claim 17, wherein the composition is formulated for intravenous or intramuscular administration.

19. A composition comprising a uric acid lowering agent comprising a uricase, and/or xanthine oxidase inhibitor formulated for parenteral administration for use in suppressing hypercatabolic state, hypercoagulation, acute respiratory distress syndrome or hyperinflammation/inflammatory cascade.

20. A composition comprising a uric acid lowering agent or agents for use to effectiveness of Interferon therapy during viral infection and decrease acute kidney injury, acute cardiac injury, acute neurologic injury, or acute pancreatic or acute organ injury.

21. A method of decreasing susceptibility to bacterial sepsis secondary to viral infection comprising administering a pharmaceutically effective amount of a UALA.

22. A method of treating a subject infected with a lytic virus comprising administering a therapeutically effective amount of a UALA during or for 1, 7, 14, 30 days or more after infection with the lytic virus.

23. The method of claim 22, wherein the lytic virus is selected from the group consisting of influenza virus, coronavirus, COVID-19, MERS, SARS, and respiratory virus

24. A method comprising co-administering a UALA and interferon during or for 30 days after infection with a lytic virus.

25. The method of claim 24, wherein the lytic virus is selected from the group consisting of influenza virus, coronavirus, COVID-19, MERS, SARS.

26. A method of improving IL-1Ra antagonism of inflammation comprising administering a therapeutically effective amount of a uric acid lowering agent.

27. A method of treating a subject undergoing an interferon resistant state, the method comprising administering a therapeutically effective amount of a UALA.

28. The method of claim 27, further comprising co-administering an interferon.

29. The method of any of claims 21-28, further comprising administering an anti-oxidant.

30. The method of claim 29, wherein the anti-oxidant comprises vitamin C or NAC.
